(19) United States
(12) Patent Application Publication
(10) Pub. No.: US 2014/0290685 A1
(43) Pub. Date: Oct. 2, 2014

(54) HAIR TREATMENT AGENTS COMPRISING SPECIFIC POLYMERS

(71) Applicant: Henkel AG & Co. KGaA, Duesseldorf (DE)

(72) Inventors: Marlene Battermann, Asendorf (DE); Christin Mueller-von Borstel, Stade (DE); Elisabeth Poppe, Hamburg (DE)

(21) Appl. No.: 14/302,592

(22) Filed: Jun. 12, 2014

Related U.S. Application Data
(63) Continuation of application No. PCT/EP2012/072255, filed on Nov. 9, 2012.

(30) Foreign Application Priority Data
Dec. 14, 2011 (DE) 10 2011 088 558.7

(51) Int. Cl. A61K 8/64; A61K 8/41; A61Q 5/02; A61K 8/73; A61K 8/37; A61Q 5/12

(52) U.S. Cl. CPC A61K 8/64; A61K 8/37; A61Q 5/12; A61Q 5/02; A61K 8/731; A61K 8/416
USPC 132/202; 424/70.122

(57) ABSTRACT

Hair treatment agents include at least one propylene glycol diester and specific polymers based on protein hydrolysates. A composition for the treatment of keratin fibers includes, in a cosmetic carrier, a total of 0.01 to 5.0 wt. %, relative to the entire composition, of at least one propylene glycol diester of formula (I), in which R1 and R2 independently represent a group selected from among at least one linear saturated or unsaturated, branched saturated or unsaturated, and cyclic saturated or unsaturated C6 to C 30 hydrocarbon group and/or a phenyl or a benzyl group optionally substituted with C1 to C4 alkyl groups and/or hydroxy groups and mixtures thereof, and a total of 0.001 to 5.0 wt. %, relative to the entire composition, of at least one polymer, said polymer being composed of at least one protein hydrolysate and at least one silane.

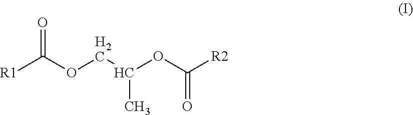

### FIELD OF THE INVENTION

The present invention generally relates to hair treatment agents comprising at least one diester of propylene glycol and specific polymers based on protein hydrolysates as well as the use of these agents for treating hair.

### BACKGROUND OF THE INVENTION

The significance of care products with a long-lasting effect is growing due not least to the serious stressing of the hair by dyeing or by permanent waving, but also to shampooing and harmful environmental factors.

However, the known active components cannot adequately meet all the requirements. Accordingly, there still remains a need for active substances or combinations of active substances for cosmetics with good caring properties. Customary silicones, in spite of their known good care properties, are viewed critically. Frequently their use is avoided when possible. Therefore there exists the great problem of at least matching and replacing the positive characteristics of the silicone oils in the cosmetic compositions, particularly for applications on keratinic fibers, such that the consumers in no case remark the difference to their habitual compositions. Ideally, conventional silicones, such as Dimethicones, Cyclomethicones and others should be entirely avoided.

EP 2025318 A1 discloses two-phase hair treatment agents comprising arylated silicones and propylene glycol dibenzoate. Hair treatment agents as macroscopically visible two- and multi-phase systems of this type are also described in the published applications EP 2025317 A1, EP 2036534 A1, EP 2022465 A1, EP 2022466 A1 and EP 2022467 A1. However, macroscopically single-phase systems comprising the combination according to the invention are described in none of these published applications.

Desirable features and characteristics of the present invention will become apparent from the subsequent detailed description of the invention and the appended claims, taken in conjunction with the accompanying drawings and this background of the invention.

### BRIEF SUMMARY OF THE INVENTION

It has now been surprisingly found that at least one diester of propylene glycol and at least one polymer based on a protein hydrolysate, particularly in the presence of additional selected quaternary compounds, afford stable effective compositions.

At the same time the use of these combinations leads to surprisingly good properties of the treated keratinic fibers, in particular to a significantly improved gloss and an increased conservation of the care and the gloss after several hair washes. The increased care performance includes improved combability, an improved hair structure, an improved elasticity as also a significantly increased wash resistance of the dyed hair, as well as a longer conservation for a concomitantly better styling performance in waving processes such as water waving and permanent waving.

These positive characteristics are obtained both with a typical rinse-off as well as with a leave-on application as well as in shampoos. In addition to the classical cream formulations, the composition according to the invention is also outstandingly suitable for spray applications, both as a non-aerosol as well as an aerosol. An exceedingly uniform spray pattern is achieved compared with the hitherto conventional products. In this way the total composition is very uniformly dispersed on the hair.

A cosmetic composition for the care of keratinic fibers comprising—each relative to the total composition—a) at least one diester of the propylene glycol according to the Formula I

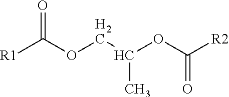

in which R1 and R2 each independently of one another stand for a residue selected from at least one linear, saturated or unsaturated, branched, saturated or unsaturated as well as cyclic saturated or unsaturated C6 to C30 hydrocarbon residue and/or a phenyl or benzyl residue that is optionally substituted with C1 to C4 alkyl groups and/or hydroxy groups as well as their mixtures in a total amount of 0.01 to 5.0 wt % and b) at least one polymer in a total amount of 0.001 to 5.0 wt %, wherein said polymer is composed of at least one protein hydrolysate as well as at least one silane.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present invention is a composition for treating keratinic fibers which, in a cosmetic carrier, comprises—each relative to the total composition—
a) at least one diester of the propylene glycol according to the Formula I

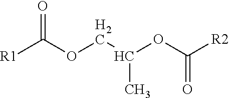

in which R1 and R2 each independently of one another stand for a residue selected from at least one linear, saturated or unsaturated, branched, saturated or unsaturated as well as cyclic saturated or unsaturated C6 to C30 hydrocarbon residue and/or a phenyl or benzyl residue that is optionally substituted with C1 to C4 alkyl groups and/or hydroxy groups as well as their mixtures in a total amount of 0.01 to 5.0 wt % and
b) at least one polymer in a total amount of 0.001 to 5.0 wt %, wherein said polymer is composed of at least one protein hydrolysate as well as at least one silane.

The ingredients a) and b) are described in detail below. In the following, when active substance complex (A) is mentioned, this term refers to the ingredients a) and b) that are mandatorily comprised in the agents according to the invention.

In the context of the present invention, hair treatment agents are for example hair conditioners, hair sprays, hair rinses, hair cures, hair masks, hair tonics, hair setting products, hair setting preparations, hair-care products, blow-dry wave lotions, foam setting products, hair gels, hair waxes or their combinations. Preferred compositions according to the invention are shampoos, conditioners or hair tonics.

Combability is inventively understood to mean both the combability of wet fibers as well as the combability of the dry fibers. The work needed for combing or the force needed during the combing procedure of a fiber bundle serves as a measure of the combability. The measurement parameters can be assessed sensorially by the person skilled in the art or quantified with measuring equipment.

The feel is defined by the tactility of a fiber bundle, wherein the person skilled in the art sensorially feels and assesses the parameters fullness and suppleness of the bundle.

Styling is understood to mean the capability to change the shape of a bundle of previously treated keratin-including fibers, especially human hair. In hair cosmetics, one may also speak of hairstyling.

Inventively suitable cosmetic carriers are particularly O/W, W/O and W/O/W emulsions in the form of creams or gels or other preparations that are particularly suitable for use on hair. In particular, the cosmetic carriers can be aqueous or aqueous-alcoholic.

An aqueous cosmetic carrier comprises at least 50 wt % water.

In the context of the present invention, aqueous alcoholic cosmetic carriers are understood to mean aqueous solutions comprising 3 to 70 wt % of a C₁-C₆ alcohol, in particular, ethanol or propanol, isopropanol, butanol, isobutanol, tert-butanol, n-pentanol, iso-pentanols, n-hexanol, iso-hexanols, glycol, glycerin, 1,2-pentane diol, 1,5-pentane diol, 1,2-hexane diol or 1,6-hexane diol. The agents according to the invention can additionally comprise further organic solvents, such as, for example, methoxybutanol, benzyl alcohol, ethyl diglycol or 1,2-propylene glycol. Preference here is given to all water-soluble organic solvents.

The cosmetic compositions comprise at least one diester of 1,2-propylene glycol of the Formula I as the ingredient a)

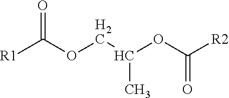

In this Formula, R1 and R2 each stand independently of one another for:

a residue selected from at least one linear, saturated or unsaturated, branched, saturated or unsaturated as well as cyclic saturated or unsaturated C6 to C30 hydrocarbon residue and/or a phenyl or benzyl residue that is optionally substituted with C1 to C4 alkyl groups and/or hydroxy groups as well as their mixtures.

Particularly preferred compounds are those, in which R1 and R2 stand for: hexyl, isohexyl, heptyl, isoheptyl, octyl, isooctyl, decyl, lauryl, myristyl, cetyl, stearyl, arachidyl, behenyl as well as the cyclic residues:

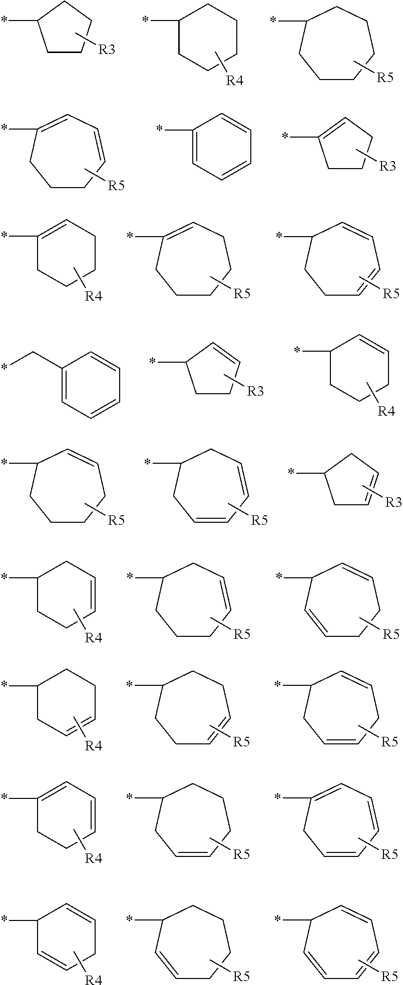

wherein the R3, R4 and R5 residues each independently of one another can stand for a hydrocarbon residue that includes one to 4 carbon atoms, a hydroxymethyl group or a hydroxy group. The star in the formulas does not stand for an atom, rather for the bonding site, at which the respective residue is connected as the R1 and/or R2 to the base molecule of Formula I. The residues R1 and R2 in Formula I are quite particularly preferably identical. The residues R1 and R2 are most preferably the above described cyclic residues.

R1 and/or R2 are most highly preferably phenyl and/or benzyl. In particular, the compound according to Formula I is propylene glycol dibenzoate.

Mixtures of compounds of Formula I may of course also be inventively used.

The compounds of Formula I are used in the cosmetic compositions in a total amount of 0.01 to 5.0 wt %, preferably 0.01 to 4.0 wt %, particularly preferably 0.05 to 3.5 wt % and most preferably 0.05 to 3.0 wt %, each relative to the total composition.

The second essential ingredient b) in the active substance complex according to the invention is a polymer that is composed of at least one protein hydrolysate as well as at least one silane.

According to the invention, the added protein hydrolysates can be of both vegetal as well as animal or marine or synthetic origin.

Animal protein hydrolysates are, for example, protein hydrolysates of elastin, collagen, keratin, silk and milk albumin, which can also be present in the form of their salts.

Furthermore, inventively preferred vegetal protein hydrolysates are for example soya, almond, pea, rice, tomato, potato and wheat protein hydrolysates.

Additional inventively preferred protein hydrolysates are of marine origin. These include for example collagen hydrolysates of fish or algae as well as protein hydrolysates of mussels or pearl hydrolysates.

Likewise, instead of protein hydrolysates, individual amino acids can also be used as the monomeric building blocks in the polymers according to the invention. In principle all amino acids can be used. Arginine, histidine, proline, cystine or lanthionine for example are preferred.

The inventive polymers of protein hydrolysates and silanes are graft copolymers, in which the silanes are grafted through organofunctional groups, for example —OH groups, onto the amino-function of the protein hydrolysate. The following Formula II depicts a silanol-terminated end of such a polymer:

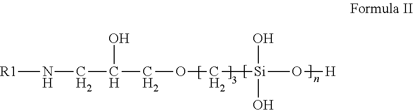

The residue R1 in this Formula stands for any polypeptide chain of the employed protein hydrolysate. The increment n stands for whole numbers from 1 to 100, preferably 1 to 50, particularly preferably 1 to 20, quite particularly preferably 1 to 10, highly preferably 1 to 5 and most highly preferably from 1 to 3.

Due to the reaction between the silanes and the amino-functions of the protein hyrolysate, the polymers according to the invention also further comprise structures of Formula III in addition to the structures of Formula II. The structure of Formula III represents a siloxane-protein hydrolysate that is crosslinked by silane. Ultimately this crosslinking can of course occur through all four silane functions. However, due to the quite considerable steric shielding in the respective reactions between the silane function and amino function of the protein hydrolysate, not all silane functions react, such that after the reaction has been terminated, siloxanes are formed that preferably include two or three siloxane-bridged branches of the protein hydrolysates. This is made clear from the Formula III.

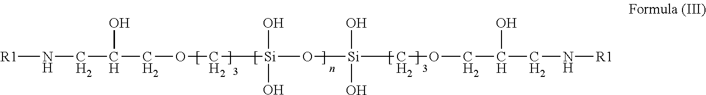

The meanings of the residues R1 and of the increment n are identical to those already described in the Formula II. These types of products are commercially available for example from Croda under the trade names Crodasone® W, Crodasone® W PF, Keravis® PE or Crodasone® Cystine.

The compositions comprise the polymers as described above in concentrations of 0.001 wt % to 5.0 wt %, preferably 0.001 wt % up to 3.0 wt %, quite particularly preferably in amounts of 0.01 wt % up to 3.0 wt %, most preferably in amounts of 0.01 up to 3.0 wt % and most highly preferably in amounts of 0.05 to 2.0 wt %.

In a second preferred embodiment of the present invention, the effect is further increased by the presence of a selected compound that includes a quaternary ammonium group.

Quaternary ammonium compounds are principally monomeric cationic or amphoteric ammonium compounds, monomeric amines, amino amides, polymeric cationic ammonium compounds as well as polymeric amphoteric ammonium compounds. From this plurality of possible quaternary ammonium compounds, the following groups have proven to be particularly suitable and are each employed as such in an amount of 0.1 to 10.0 wt %. This quantity is also not less nor exceeded if a mixture of different compounds of the quaternary ammonium compounds is used.

Esterquats according to the Formula (Tkat1-2) form the first group.

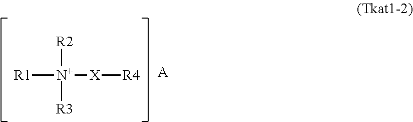

in which the R1, R2 and R3 residues are each independent of one another and may be the same or different. The R1, R2 and R3 residues mean:
a branched or unbranched alkyl residue that includes 1 to 4 carbon atoms which may comprise at least one hydroxy group, or
a saturated or unsaturated, branched or unbranched or a cyclic saturated or unsaturated alkyl residue that includes 6 to 30 carbon atoms which may comprise at least one hydroxy group, or
an aryl or alkaryl residue, for example phenyl or benzyl,
the residue (—X—R4), with the proviso that at most 2 of the R1, R2 or R3 residues may stand for this residue:

The residue (—X—R4) is comprised at least 1 to 3 times.

Herein X stands for:
1) —(CH₂)ₙ— with n=1 to 20, preferably n=1 to 10 and particularly preferably n=1-5, or
2) —(CH₂—CHR5-O)n- with n=1 to 200, preferably 1 to 100 and particularly preferably 1 to 50 with R5 meaning hydrogen, methyl or ethyl,
3) a hydroxyalkyl group that includes one to four carbon atoms which can be branched or unbranched, and which comprises at least one and at most 3 hydroxy groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues,
   and R4 stands for:
1) R6-O—CO—, in which R6 is a saturated or unsaturated, branched or unbranched or a cyclic saturated or unsaturated alkyl residue that includes 6 to 30 carbon atoms which may comprise at least one hydroxy group, and which optionally may be further oxethylated with 1 to 100 ethylene oxide units and/or 1 to 100 propylene oxide units, or
2) R7-CO—, in which R7 is a saturated or unsaturated, branched or unbranched or a cyclic saturated or unsaturated alkyl residue that includes 6 to 30 carbon atoms which may comprise at least one hydroxy group, and which optionally may be further oxethylated with 1 to 100 ethylene oxide units and/or 1 to 100 propylene oxide units,
   and A stands for a physiologically acceptable organic or inorganic anion and is defined here to also represent all A in the structures described hereinafter. The anion of all the described cationic compounds is selected from the halide ions, fluoride, chloride, bromide, iodide, sulfates of the general Formula RSO₃⁻, in which R means saturated or unsaturated alkyl residues that includes 1 to 4 carbon atoms, or anionic residues of organic acids such as maleate, fumarate, oxalate, tartrate, citrate, lactate or acetate.

Such products are marketed, for example, under the trade names Rewoquat®, Stepantex®, Dehyquart®, Armocare® and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these esterquats.

Further inventively particularly preferred compounds of the Formula (Tkat1-2) include the cationic betaine esters of the Formula (Tkat1-2.1).

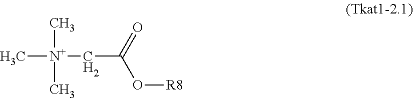

R8 corresponds to the meaning of R7.

The esterquats with the trade names Armocare® VGH-70, as well as Dehyquart® F-75, Dehyquart® L80, Stepantex® VS 90 and Akypoquat® 131 are particularly preferred.

Quaternary imidazoline compounds are another group. The structure of these compounds is shown below in the illustrated Formula (Tkat2).

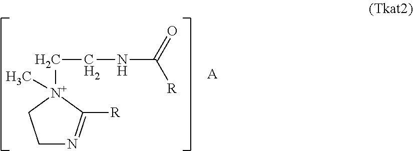

The R residues independently of each other each stand for a saturated or unsaturated, linear or branched hydrocarbon residue with a chain length of 8 to 30 carbon atoms. The preferred compounds of the Formula (Tkat2) each comprise the same hydrocarbon residue for R. The chain length of the R residue is preferably 12 to 21 carbon atoms. A stands for an anion as described above. Particular inventive examples are available for example under the INCI names Quaternium-27, Quaternium-72, Quaternium-83 and Quaternium-91. According to the invention, Quaternium-91 is most highly preferred.

In a particularly preferred embodiment of the invention, the agents according to the invention further comprise at least one amine and/or cationized amine, in particular an amido amine and/or a cationized amido amine with the following structural formula:

R1-NH—(CH₂)ₙ—N⁺R²R³R⁴A (Tkat3)

in which R1 can be an acyl or alkyl residue that includes 6 to 30 atoms which can be branched or unbranched, saturated or unsaturated, and wherein the acyl residue and/or the alkyl residue can comprise at least one OH group, and
R2, R3 and R4 independently of each other can be
1) hydrogen or
2) an alkyl residue that includes 1 to 4 carbon atoms which can be the same or different, saturated or unsaturated, and
3) a branched or unbranched hydroxyalkyl group that includes one to 4 carbon atoms with at least one and at most three hydroxy groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues, and
   A is an anion as described above and
   n means a whole number between 1 and 10.

A preferred composition is one, in which the amine and/or the quaternized amine according to the general Formula (Tkat3) is an amido amine and/or a quaternized amido amine, in which R1 means a branched or unbranched, saturated or unsaturated acyl residue that includes 6 to 30 carbon atoms which can comprise at least one OH group. In this regard, a fatty acid residue from oils and waxes, in particular from naturally occurring oils and waxes, is preferred. Lanolin, beeswax or candellila waxes are examples of these.

Those amido amines and/or quaternized amido amines are also preferred in which R2, R3 and/or R4 in the Formula (Tkat3) mean a residue according to the general Formula CH₂CH₂OR5, in which R5 can mean alkyl residues that includes 1 to 4 carbon atoms, hydroxyethyl or hydrogen. The preferred value of n in the general Formula (Tkat8) is a whole number between 2 and 5.

The alkylamido amines can both be present as such and be converted by protonation in appropriately acidic solution into a quaternary compound in the composition. The cationic alkylamido amines are inventively preferred.

Examples of such commercial products according to the invention are Witcamine® 100, Incromine® BB, Mackine® 401 and other Mackine® types, Adogen® S18V, and as the permanently cationic amido amines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl HE.

Additional quaternary ammonium compounds are ammonium halides, particularly chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, behenyltrimethylammonium chloride, distearyldimethylammonium chloride, lauryldimethylammonium chloride, lauryldimethylbenzylammonium chloride and tricetylmethylammonium chloride. The long alkyl chains of the abovementioned surfactants have preferably 10 to 22 carbon atoms. The abovementioned cationic surfactants can be used individually or together in any combination, wherein amounts between 0.01 to 10 wt %, preferably in amounts of 0.01 to 7.5 wt % and quite particularly preferably in amounts of 0.1 to 5.0 wt % are comprised. In this regard, the best results of all are obtained with amounts of 0.1 to 3.0 wt %, each relative to the total composition of the relevant agent.

Other quaternary ammonium compounds are cationic and amphoteric polymers.

The cationic and/or amphoteric polymers can be homopolymers or copolymers or polymers based on naturally occurring polymers, wherein the quaternary nitrogen groups are comprised either in the polymer chain or preferably as a substituent on one or more of the monomers. The ammonium group-including monomers can be copolymerized with non-cationic monomers. Suitable cationic monomers are unsaturated, radical polymerizable compounds that carry at least one cationic group, in particular ammonium substituted vinyl monomers, such as for example trialkylmethacryloxyalkylammonium, trialkylacryloxyalkylammonium, dialkyldiallylammonium and quaternary vinylammonium monomers that includes cyclic, cationic nitrogen-including groups, such as pyridinium, imidazolium or quaternary pyrrolidones, e.g. alkylvinylimidazolium, alkylvinylpyridinium, or alkylvinylpyrrolidone salts. The alkyl groups of these monomers are preferably lower alkyl groups, such as for example C1 to C7 alkyl groups, particularly preferably C1 to C3 alkyl groups.

The ammonium group-including monomers can be copolymerized with non-cationic monomers. Exemplary suitable comonomers are acrylamide, methacrylamide, alkyl and dialkylacrylamide, alkyl and dialkylmethacrylamide, alkyl acrylate, alkyl methacrylate, vinyl caprolactone, vinyl caprolactam, vinyl pyrrolidone, vinyl esters, e.g. vinyl acetate, vinyl alcohol, propylene glycol or ethylene glycol, wherein the alkyl groups of these monomers are preferably C1 to C7 alkyl groups, particularly preferably C1 to C3 alkyl groups.

From the plurality of these polymers, particularly effective ingredients of the active substance complex according to the invention have proven to be: Homopolymers of the general Formula —{CH₂—[CR¹COO—(CH₂)ₘN⁺R²R³R⁴]}ₙX⁻, in which R¹=—H or —CH₃, R², R³ and R⁴ independently of each other are selected from C1-4 alkyl, -alkenyl or -hydroxyalkyl groups, m=1, 2, 3 or 4, n is a natural number and X⁻ is a physiologically acceptable organic or inorganic anion. Regarding these polymers, those that are preferred in accordance with the invention meet at least one of the following conditions: R¹ stands for a methyl group, R², R³ and R⁴ stand for methyl groups, m has the value 2.

Exemplary physiologically acceptable counter ions X⁻ include halide ions, sulfate ions, phosphate ions, methosulfate ions as well as organic ions such as lactate, citrate, tartrate and acetate ions. Methosulfate and halide ions are preferred, particularly chloride.

Suitable cationic polymers that are derived from synthetic polymers are for example copolymers of A1) comprising 0.1 to 50%, preferably 10 to 50% of monomers of the Formula (Ia) (relative to the total number of monomers in the copolymer)

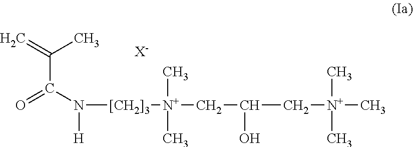

in which X stands for chloride, sulfate, methosulfate, and

A2) monomers from the group acrylic acid, methacrylic acid as well as the alkali metal and ammonium salts of these acids, wherein the monomer A2 makes up 50 to 99.9%, preferably 50 to 90% of the copolymer (relative to the total number of monomers in the copolymer).

A most highly preferred polymer that has the structure as illustrated above is commercially available under the name Polyquaternium-74.

A particularly suitable homopolymer is the optionally crosslinked poly(methacryloyloxyethyltrimethylammonium chloride) with the INCI name Polyquatemium-37. Such products are commercially available for example under the trade names Rheocare® CTH (Cosmetic Rheologies) and Synthalen® CR (3V Sigma).

The homopolymer is preferably employed in the form of a non-aqueous polymer dispersion. Such polymer dispersions are commercially available under the trade names Salcare® SC 95 and Salcare® SC 96.

Suitable cationic polymers derived from naturally occurring polymers are cationic derivatives of polysaccharides, for example cationic derivatives of cellulose, starch or guar. Chitosan and chitosan derivatives are also suitable. Cationic polysaccharides have the general Formula G-O—B—N+RₐR_{b}R_{c}A⁻

G is an anhydroglucose residue, for example starch- or cellulose-anhydroglucose;
B is a divalent linking group, for example alkylene, oxyalkylene, polyoxyalkylene or hydroxyalkylene;
Rₐ, R_{b} and R_{c} independently of each other are alkyl, aryl, alkylaryl, arylalkyl, alkoxyalkyl or alkoxyaryl, each including up to 18 carbon atoms, wherein the total number of the carbon atoms in Rₐ, R_{b} and R_{c} is preferably 20 at most;
A⁻ is a typical counter anion and is preferably chloride.

Cationic, i.e. quaternized celluloses, are commercially available with different degrees of substitution, cationic charge density, nitrogen content and molecular weights. For example, Polyquaternium-67 is commercially available under the trade names Polymer® SL or Polymer® SK (Amerchol). Another most highly preferred cellulose is commercially available from Croda under the trade name Mirustyle® CP. This is a Trimonium and Cocodimonium Hydroxyethylcellulose as a derivatized cellulose with the INCI name Polyquaternium-72. Polyquaternium-72 can be used in solid form and also in pre-dissolved form in aqueous solution.

Additional cationic celluloses are available under the names Polymer JR® 400 (Amerchol, INCI name Polyquaternium-10) and Polymer Quatrisoft® LM-200 (Amerchol, INCI name Polyquaternium-24). Other commercial products are the compounds Celquat® H 100 and Celquat® L 200. Finally, another derivatized cellulose with the INCI name Polyquaternium-72 has the trade name Mirustyle® CP from Croda with Trimonium and Cocodimonium Hydroxyethylcellulose. Polyquaternium-72 can be used in solid form and also in pre-dissolved form in aqueous solution. Particularly preferred cationic celluloses are Polyquaternium-10, Polyquaternium-24, Polyquaternium-67 and Polyquaternium-72.

Suitable cationic guar derivatives are marketed under the trade name Jaguar® and have the INCI name Guar Hydroxypropyltrimonium Chloride. Other particularly suitable cationic guar derivatives are also commercially available from Hercules under the trade name N-Hance®. Other cationic guar derivatives are commercially available from Cognis under the trade name Cosmedia®. A preferred cationic guar derivative is the commercial product AquaCat® of Hercules. This raw material concerns an already pre-dissolved cationic guar derivative. The cationic guar derivatives are inventively preferred.

A suitable chitosan is available for example from Kyowa Oil & Fat, Japan, under the trade name Flonac®. A preferred chitosan salt is chitosonium pyrrolidone carboxylate, which is marketed for example under the name Kytamer® PC from Amerchol, USA. Other chitosan derivatives are freely available under the trade names Hydagen® CMF, Hydagen® HCMF and Chitolam® NB/101.

Finally, cationic polymers based on sugars are inventively useable with preference.

These compounds are for example cationic alkyl oligoglucosides as shown in the following Figure.

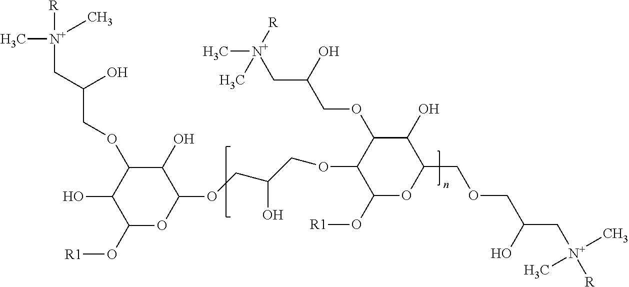

In the above formula the R residues, independently of each other, stand for a linear or branched C6 to C30 alkyl residue, a linear or branched C6-C30 alkenyl residue, preferably the R residue stands for an R residue selected from lauryl, myristyl, cetyl, stearyl, oleyl, behenyl or arachidyl.

The R1 residues, independently of each other, stand for a linear or branched C6 to C30 alkyl residue, a linear or branched C6 to C30 alkenyl residue, preferably the R residue stands for a residue selected from butyl, capryl, caprylyl, octyl, nonyl, decanyl, lauryl, myristyl, cetyl, stearyl, oleyl, behenyl or arachidyl. The R1 residues are particularly preferably the same. Even more preferably the R1 residues are selected from industrial mixtures of fatty alcohol fractions of C6/C8 fatty alcohols, C8/C10 fatty alcohols, C10/C12 fatty alcohols, C12/C14 fatty alcohols, C12/C18 fatty alcohols, and most preferably in this regard those industrial fatty alcohol fractions that are of vegetal origin.

Particularly preferred examples of cationic alkyl oligoglucosides are the compounds with the INCI names Polyquaternium-77, Polyquaternium-78, Polyquaternium-79, Polyquaternium-80, Polyquatemium-81 and Polyquaternium-82. Most highly preferred cationic alkyl oligoglucosides have the trade names Polyquaternium-77, Polyquatemium-81 and Polyquaternium-82.

Such compounds can be obtained under the name Poly Suga® Quat from Colonial Chemical Inc.

The cationic alkyl oligoglucosides are used in a total amount of 0.01 to 10.0 wt %, preferably 0.05 to 5.0 wt %, more preferably 0.1 to 3.0 wt % and most preferably in amounts of 0.2 to 2.0 wt %, each relative to the total weight of the composition. Mixtures of cationic alkyl oligoglucosides may, of course, also be inventively used. In this case it is preferred to use simultaneously a long chain and a short chain cationic alkyl oligoglucoside.

Another cationic polymer can be obtained based on ethanolamine. The polymer is commercially available under the name Polyquaternium-71.

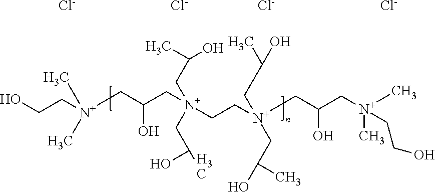

This polymer can be obtained for example under the name Cola® Moist 300P from Colonial Chemical Inc.

The Polyquaternium-71 is used in a total amount of 0.01 to 10.0 wt %, preferably 0.05 to 5.0 wt %, more preferably 0.1 to 3.0 wt % and most preferably in amounts of 0.2 to 2.0 wt %, each relative to the total weight of the composition. Further preferred cationic polymers are, for example
cationized honey, for example the commercial product Honeyquat® 50,
polymeric dimethyldiallylammonium salts and their copolymers with esters and amides of acrylic acid and methacrylic acid. The commercially available products Merquat® 100 (poly(dimethyldiallylammonium chloride)) and Merquat® 550 (dimethyldiallylammonium chloride-acrylamide copolymer) are examples of such cationic polymers with the INCI name Polyquaternium-7,
vinyl pyrrolidone-vinyl imidazolium methochloride copolymers, as are offered under the trade names Luviquat® FC 370, FC 550 and the INCI name Polyquaternium-16 as well as FC 905 and HM 552,
quaternized vinyl pyrrolidone/dimethylaminoethyl methacrylate, for example vinyl pyrrolidone/dimethylaminoethyl methacrylate methosulfate copolymer, which is marketed under the trade names Gafquat® 755 N and Gafquat® 734 by Gaf Co., USA and the INCI name Polyquatemium-11,
quaternized polyvinyl alcohol,
as well as the polymers that include quaternary nitrogen atoms in the main polymer chain, known under the names Polyquaternium-2, Polyquaternium-17, Polyquaternium-18 and Polyquaternium-27,
vinyl pyrrolidone-vinyl caprolactam-acrylate terpolymers with acrylic acid esters and acrylamides as the third monomer moieties as are commercially available, for example, under the trade name Aquaflex® SF 40.

Amphoteric polymers according to the invention are those polymers, in which a cationic group is derived from at least one of the following monomers:
(i) monomers with quaternary ammonium groups of the general Formula (Mono1),

R¹—CH═CR²—CO—Z—(CₙH₂ₙ)—N⁽⁺⁾R²R³R⁴A⁽⁻⁾ (Mono1)
in which R¹ and R² independently of each other stand for hydrogen or a methyl group and R³, R⁴ and R⁵ independently of one another for alkyl groups with 1 to 4 carbon atoms, Z for an NH-group or an oxygen atom, n for a whole number from 2 to 5 and A⁽⁻⁾ is the anion of an organic or inorganic acid,
(ii) monomers with quaternary ammonium groups of the general Formula (Mono2),

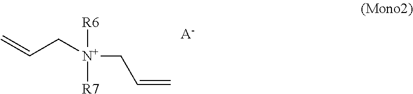

in which R⁶ and R⁷ stand independently of one another for a (C1 to C4) alkyl group, in particular for a methyl group and
A⁻ is the anion of an organic or inorganic acid,
(iii) monomeric carboxylic acids of the general Formula (Mono3),

R⁸—CH═CR⁹—COOH (Mono3)
in which R⁸ and R⁹, independently of one another are hydrogen or methyl groups.

Particularly preferred are such polymers, which incorporate monomers of type (i), in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (i). Acrylic acid is preferably used as the monomer (ii) in the cited polymers.

Particularly preferred amphoteric polymers are copolymers of at least one monomer (Mono1) or (Mono2) with the monomer (Mono3), in particular copolymers of the monomers (Mono2) and (Mono3). Inventively quite particularly preferably used amphoteric polymers are copolymers of diallyldimethylammonium chloride and acrylic acid. These copolymers are marketed under the INCI name Polyquaternium-22 inter alia with the trade name Merquat® 280 (Nalco).

Moreover, the amphoteric polymers according to the invention can additionally comprise, besides a monomer (Mono 1) or (Mono2) and a monomer (Mono3), a monomer (Mono4)

(iv) monomeric carboxylic acid amides of the general Formula (Mono4),

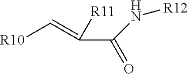

in which R¹⁰ and R¹¹ independently of one another are hydrogen or methyl groups and R¹² stands for a hydrogen atom or a (C₁- to C₈) alkyl group.

Inventively quite particularly preferably used amphoteric polymers based on a comonomer (Mono4) are terpolymers of diallyldimethylammonium chloride, acrylamide and acrylic acid. These copolymers are marketed under the INCI name Polyquatemium-39 inter alia with the trade name Merquat® Plus 3330 (Nalco).

According to the invention, the amphoteric polymers can be both added directly as well as in salt form, the latter being obtained by neutralization of the polymer with an alkali metal hydroxide, for example.

The abovementioned cationic polymers can be used individually or together in any combination, wherein amounts between 0.01 to 10 wt %, preferably in amounts of 0.01 to 7.5 wt % and quite particularly preferably in amounts of 0.1 to 5.0 wt % are comprised. In this regard, the best results of all are obtained with amounts of 0.1 to 3.0 wt %, each relative to the total composition of the relevant agent.

Furthermore, the agents according to the invention preferably comprise at least one silicone polymer selected from the group of the Dimethiconols and/or the group of the amino-functional silicones and/or the group of the Dimethicones and/or the group of the Cyclomethicones. However, the compositions according to the invention shall not comprise any arylated silicones in any of the respective groups of silicones.

The Dimethicones according to the invention can be linear as well as branched as well as cyclic or cyclic and branched. Linear Dimethicones can be represented by the following structural Formula (Si1):

(SiR¹₃)—O—(SiR²₂—O—)ₓ—(SiR¹₃) (Si1)

Branched Dimethicones can be represented by the following structural Formula (Si1.1):

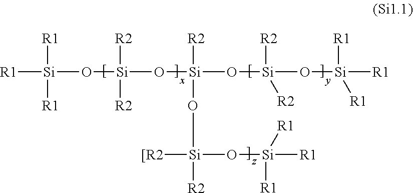

The residues R¹ and R² each stand independently of one another for hydrogen, a methyl residue, a C2 to C30 linear, saturated or unsaturated hydrocarbon residue, a phenyl residue and/or an aryl residue. The numbers x, y and z are whole numbers and each, independently of each other, range from 0 to 50.000. The molecular weights of the Dimethicones lie between 1000 Da and 10 000 000 Da. The viscosities range between 100 and 10 000 000 cPs, measured at 25° C. with a glass capillary viscosimeter following the Dow Corning Corporate Test Method CTM 0004 of 20 Jul. 1970. Preferred viscosities are between 1000 and 5 000 000 cPs, quite particularly preferred viscosities are between 10 000 and 3 000 000 cPs. The most preferred range is between 50 000 and 2 000 000 cPs. Most highly preferred viscosities are in the region of about 60 000 cPs. As an example, reference may be made to the product “Dow Corning 200 with 60 000 cSt”.

Particularly preferred cosmetic or dermatological preparations according to the invention are characterized in that they comprise at least one silicone of the Formula (Si1.2)

(CH₃)₃Si—[O—Si(CH₃)₂]ₓ—O—Si(CH₃)₃ (Si1.2),

in which x stands for a number from 0 to 100, advantageously from 0 to 50, more preferably from 0 to 20 and especially 0 to 10.

The Dimethicones (Si1) are comprised in the compositions according to the invention in amounts of 0.01 to 10 wt %, preferably 0.01 to 8 wt %, particularly preferably 0.1 to 7.5 wt % and in particular 0.1 to 5 wt %, relative to the total composition.

Finally, the silicone compounds are understood to include the Dimethiconols (Si8). The Dimethiconols according to the invention can be linear as well as branched as well as cyclic or cyclic and branched. Linear Dimethiconols can be represented by the following structural Formula (Si8-I):

(SiOHR¹₂)—O—(SiR²₂—O—)ₓ—(SiOHR¹₂) (Si8-I)

Branched Dimethiconols can be represented by the following structural formula (Si8-II):

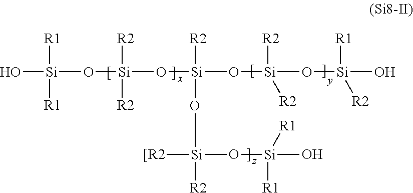

The residues R¹ and R² each stand independently of one another for hydrogen, a methyl residue, a C2 to C30 linear, saturated or unsaturated hydrocarbon residue. The numbers x, y and z are whole numbers and each, independently of each other, range from 0 to 50 000. The molecular weights of the Dimethiconols lie between 1000 Da and 10 000 000 Da. The viscosities range between 100 and 10 000 000 cPs, measured at 25° C. with a glass capillary viscosimeter following the Dow Corning Corporate Test Method CTM 0004 of 20 Jul. 1970. Preferred viscosities are between 1000 and 5 000 000 cPs, quite particularly preferred viscosities are between 10 000 and 3 000 000 cPs. The most preferred range is between 50 000 and 2 000 000 cPs.

The following commercial products are given as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four from GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH). The Dimethiconols (Si8) are comprised in the compositions according to the invention in amounts of 0.01 to 10 wt %, preferably 0.01 to 8 wt %, particularly preferably 0.1 to 7.5 wt % and in particular 0.1 to 5 wt % of Dimethiconol, relative to the composition.

Particularly preferred agents according to the invention comprise one or more amino-functional silicones. Such silicones can be described, for example, by the Formula (Si-2)

M(RₐQ_{b}SiO_{(4-a-b)/2)x}(R_{c}SiO_{(4-c)/2)y}M (Si-2)

wherein, in the above formula
R is a hydrocarbon or a hydrocarbon residue that includes 1 to about 6 carbon atoms,
Q is a polar residue of the general Formula —R¹HZ,
   in which
   R¹ is a divalent linking group that is bonded to hydrogen and the Z residue, composed of carbon and hydrogen atoms, carbon, hydrogen and oxygen atoms or carbon, hydrogen and nitrogen atoms, and
   Z is an organic, amino-functional residue that comprises an amino-functional group;
a assumes values in the range of about 0 to about 2,
b assumes values in the range of about 1 to about 3,
a+b is less than or equal to 3, and
c is a number in the range of about 1 to about 3, and
x is a number in the range of 1 to about 2000, preferably from about 3 to about 50 and most preferably from about 3 to about 25, and
y is a number in the range of about 20 to about 10 000, preferably from about 125 to about 10 000 and most preferably from about 150 to about 1000, and
M is a suitable silicone end group, as is known from the prior art, preferably trimethylsiloxy.
   Z according to Formula (Si-2) is an organic, amino-functional residue comprising at least one functional amino group. A possible formula for said Z is NH(CH₂)_{z}NH₂, in which z stands for a whole number greater than or equal to 1. Another possible formula for said Z is —NH(CH₂)_{z}(CH₂)_{zz}NH, in which both z and also zz independently of one another are a whole number greater than or equal to 1, wherein this structure includes diamino ring structures, such as piperazinyl. Most preferably, said Z is an —NHCH₂CH₂NH₂ residue. Another possible formula for said Z is —N(CH₂)_{z}(CH₂)_{zz}NX₂ or —NX₂, in which each X of X₂ is independently selected from the group consisting of hydrogen and alkyl groups with 1 to 12 carbon atoms, and zz is 0.
   Q according to Formula (Si-2) is most preferably a polar amino-functional residue of the Formula —CH₂CH₂CH₂NHCH₂CH₂NH₂.

In the Formula (Si-2), a assumes values in the range 0 to 2, b assumes values in the range 2 to 3, a+b is less than or equal to 3, and c is a number in the range 1 to 3. Cationic silicone oils that are inventively suitable are for example the commercially available products Dow Corning (DC) 929 Emulsion, DC2-2078, DC5-7113, SM-2059 (General Electric) and SLM-55067 (Wacker).

Particularly preferred inventive agents are characterized in that they comprise at least one amino-functional silicone of the Formula (Si3-a)

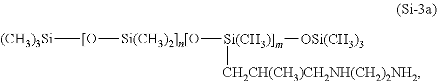

in which m and n are numbers whose sum (m+n) is between 1 and 2000, preferably between 50 and 150, wherein n preferably assumes values of 0 to 1999 and particularly from 49 to 149 and m preferably assumes values of 1 to 2000, particularly 1 to 10.

These silicones are called Trimethylsilylamodimethicone according to the INCI declaration and are available for example under the name Q2-7224 (manufacturer: Dow Corning; a stabilized Trimethylsilylamodimethicone).

Particularly preferred inventive agents are also those that comprise at least one amino-functional silicone of the formula (Si-3b)

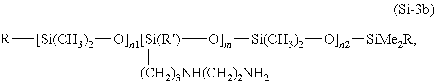

in which
R stands for —OH, an (optionally ethoxylated and/or propoxylated) C₁ to C₂₀ alkoxy group or a —CH₃ group,
R′ stands for —OH, a C₁ to C₂₀ alkoxy group or a —CH₃ group and
m, n1 and n2 are numbers, whose sum (m+n1+n2) is between 1 and 2000, preferably between 50 and 150, wherein the sum (n1+n2) preferably assumes values of 0 to 1999 and particularly from 49 to 149 and m preferably assumes values of 1 to 2000, particularly 1 to 10.

In accordance with the INCI Declaration, these silicones are called an Amodimethicone or a functionalized Amodimethicone, such as for example Bis(C13-15 Alkoxy) PG Amodimethicone (available for example as the commercial product: DC 8500 from Dow Corning), Trideceth-9 PG-Amodimethicone (available as the commercial product Silcare Silicone SEA from Clariant). Suitable diquaternary silicones are selected from compounds of the general Formula (Si3c))

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

wherein the R1 to R6 residues independently of each other mean C1 to C22 alkyl residues that can comprise hydroxy groups and wherein preferably at least one of the residues possesses at least 8 carbon atoms and the other residues possess 1 to 4 carbon atoms, the R7 to R12 residues independently of each other are the same or different and mean C1 to C10 alkyl, A means a divalent organic linking group,
n is a number from 0 to 200, preferably from 10 to 120, particularly preferably from 10 to 40, and X⁻ is an anion.
The divalent linking group is preferably a C1 to C12 alkylene or alkoxyalkylene group that can be substituted with one or more hydroxy groups. The —(CH₂)₃—O—CH₂—CH(OH)—CH₂— group is particularly preferred.
The anion X⁻ can be a halide ion, an acetate, an organic carboxylate or a compound of the general Formula RSO₃⁻, in which R means a C1 to C4 alkyl residue.

A preferred diquaternary silicone has the general Formula (Si3d)

[RN⁺Me₂-A-(SiMe₂O)ₙ—SiMe₂-A-N⁺Me₂R]2CH₃COO⁻ (Si3d),

wherein A is the group —(CH₂)₃—O—CH₂—CH(OH)—CH₂—,
R is an alkyl residue that includes at least 8 carbon atoms and n is a number from 10 to 120.

Suitable silicone polymers that include two terminal, quaternary ammonium groups are known under the INCI name Quaternium-80. This refers to dimethylsiloxanes that include two terminal trialkylammonium groups. Such diquaternary polydimethylsiloxanes are marketed by Evonik under the trade names Abil® Quat 3270, 3272 and 3474.

Inventively preferred hair treatment agents are characterized in that they comprise, based on their weight, 0.01 to 10 wt %, preferably 0.01 to 8 wt %, particularly preferably 0.1 to 7.5 wt % and in particular 0.2 to 5 wt % aminofunctional silicone(s) and/or diquaternary silicone.

Polyammonium-polysiloxane compounds are another amino-functional silicone according to the invention. The polyammonium-polysiloxane compounds can be obtained for example under the trade name Baysilone® from GE Bayer Silicones. In this regard, the products with the trade names Baysilone TP 3911, SME 253 and SFE 839 are preferred. The use of Baysilone TP 3911 is quite particularly preferred as the active component of the composition according to the invention. The polyammonium-polysiloxane compounds are used in the compositions according to the invention in an amount of 0.01 to 10 wt %, preferably 0.01 to 7.5 wt %, particularly preferably 0.01 to 5.0 wt %, quite particularly preferably 0.05 to 2.5 wt % each relative to the total composition.

EP 1887024 A1 describes novel cationic amino-functional silicones that improve in particular the gloss in agents for the care of surfaces, for example human hair. These cationic silicone polymers are characterized in that they possess a silicone backbone as well as at least one polyether moiety and additionally at least one moiety with an ammonium structure. In the context of the present invention, besides the compounds of the abovementioned EP 1887024 A1, the exemplary preferred cationic silicone polymers are in particular the compounds with the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22 as well as Silicone Quaternium-2 Panthenol Succinate and Silicone Quaternium-16/Glycidyl Dimethicone Crosspolymer. Silicone Quaternium-22 in particular is the most preferred. This raw material is marketed for example by Evonik under the trade name Abil® T-Quat 60.

The cationic amino-functional silicone polymers are comprised in the compositions according to the invention in amounts of 0.01 to 20 wt %, preferably in amounts of 0.05 to 10 wt % and quite particularly preferably in amounts of 0.1 to 7.5 wt %. In this regard, the best results of all are obtained with amounts of 0.1 to 5 wt %, each relative to the total composition of the relevant agent.

The addition of cyclic Dimethicones, designated by INCI as Cyclomethicone, is also inventively preferred. Here, preferred cosmetic or dermatological preparations according to the invention comprise at least one silicone of the Formula (Si-4)

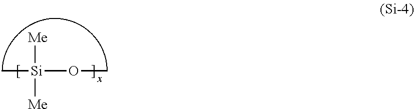

in which x stands for a number from 3 to 200, advantageously from 3 to 10, more preferably from 3 to 7 and especially 3, 4, 5 or 6.

Likewise inventively preferred agents are characterized in that they comprise at least one silicone of the Formula (Si-5)

R₃Si—[O—SiR₂]ₓ—(CH₂)ₙ—[O—SiR₂]_{y}—O—SiR₃ (Si-5),

in which R stands for the same or different residues from the group —H, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x or y stand for a number from 0 to 200, preferably from 0 to 10, more preferably from 0 to 7 and especially 0, 1, 2, 3, 4, 5 or 6, and n stands for a number from 0 to 10, preferably from 1 to 8 and particularly for 2, 3, 4, 5, 6.

Besides the Dimethicones, Dimethiconols, Amodimethicones and/or Cyclomethicones according to the invention, the compositions according to the invention can comprise water-soluble silicones as additional silicones.

Suitable hydrophilic silicones are selected for example from the compounds of the Formulas (Si-6) and/or (Si-7). Particularly preferred water-soluble surfactants based on silicone are selected from the group of the Dimethicon copolyols that are preferably alkoxylated, especially polyethoxylated or polypropoxylated.

Dimethicon copolyols are understood to mean inventively preferred polyoxyalkylene-modified dimethylpolysiloxanes of the general Formulas (Si-6) or (Si-7):

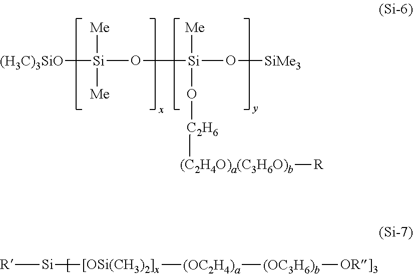

in which the R residue stands for a hydrogen atom, an alkyl group that includes 1 to 12 carbon atoms, an alkoxy group that includes 1 to 12 carbon atoms or a hydroxy group; the R′ and R″ residues mean alkyl groups that include 1 to 12 carbon atoms, x stands for a whole number from 1 to 100, preferably from 20 to 30, y stands for a whole number from 1 to 20, preferably from 2 to 10 and a and b stand for whole numbers from 0 to 50, preferably from 10 to 30.

In the context of the invention, particularly preferred Dimethicon copolyols are for example the commercially marketed products under the trade names SILWET® (Union Carbide Corporation) and DOW CORNING. Inventively particularly preferred Dimethicon copolyols are Dow Corning 190 and Dow Corning 193.

The Dimethicon copolyols are comprised in the compositions according to the invention in amounts of 0.01 to 10 wt %, preferably 0.01 to 8 wt %, particularly preferably 0.1 to 7.5 wt % and in particular 0.1 to 5 wt % of Dimethicon copolyol, relative to the composition.

Another care ingredient that can be particularly preferably used is an ester oil. In the preferred compositions according to the invention, preference is given to ester oils over the silicone oils. The ester oils are defined as follows:

Ester oils are understood to mean the esters of C₆-C₃₀ fatty acids with C₂-C₃₀ fatty alcohols. The monoesters of fatty acids with alcohols that include 2 to 24 carbon atoms are preferred. Examples of the fatty acid moieties in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Examples of the fatty alcohol moieties in the ester oils are isopropyl alcohol, capronyl alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linalyl alcohol, linolenyl alcohol, elaeostearyl alcohol, arachyl alcohol, gadoleyl alcohol, behenyl alcohol, erucyl alcohol and brassidyl alcohol as well as their industrial mixtures. According to the invention, isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerin tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.

The ester oils can of course also be alkoxylated with ethylene oxide, propylene oxide or mixtures of ethylene oxide and propylene oxide. In this regard, the alkoxylation can be on the fatty alcohol moiety as well as on the fatty acid moiety as well as on both parts of the ester oils. However, it is inventively preferred if the fatty alcohol was first alkoxylated and then esterified with fatty acid. These compounds are generally illustrated in the Formula (D4-II).

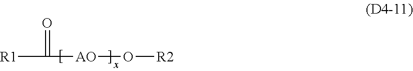

R1 stands here for a saturated or unsaturated, branched or unbranched, cyclic saturated cyclic unsaturated acyl residue that includes 6 to 30 carbon atoms,
AO stands for ethylene oxide, propylene oxide or butylene oxide,
X stands for a number between 1 and 200, preferably 1 and 100, particularly preferably between 1 and 50, quite particularly preferably between 1 and 20, highly preferably between 1 and 10 and most preferably between 1 and 5,
R2 stands here for a saturated or unsaturated, branched or unbranched, cyclic saturated cyclic unsaturated alkyl, alkenyl, alkynyl, phenyl or benzyl residue that includes 6 to 30 carbon atoms. Examples of the fatty acid moieties employed as the R1 residue in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Examples of the fatty alcohol moieties in the ester oils are isopropyl alcohol, capronyl alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linalyl alcohol, linolenyl alcohol, elaeostearyl alcohol, arachyl alcohol, gadoleyl alcohol, behenyl alcohol, erucyl alcohol and brassidyl alcohol as well as their industrial mixtures. An inventively particularly preferred ester oil is available for example under the INCI name PPG-3 Benzyl Ether Myristate.

Additionally understood as ester oils are:
dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate,
as well as symmetrical, unsymmetrical or cyclic esters of carbon dioxide with fatty alcohols, e.g. glycerin carbonate or dicaprylyl carbonate (Cetiol® CC),
trifatty acid esters of saturated and/or unsaturated linear and/or branched fatty acids with glycerin,
fatty acid partial glycerides, under which are understood monoglycerides, diglycerides and their industrial mixtures. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably employed.

The ester oils are used in the agents according to the invention in an amount of 0.01 to 20 wt %, preferably 0.01 to 10.0 wt %, particularly preferably 0.01 to 7.5 wt %, highly preferably 0.1 to 5.0 wt %. Of course it is inventively possible to also use a plurality of ester oils at the same time.

Other oily bodies according to the invention are:
vegetal oils. Examples of such oils are argan oil, apricot kernel oil, marula oil, macadamia oil, sesame oil, sunflower oil, olive oil, soya oil, rapeseed oil, almond oil, jojoba oil, orange oil, cape chestnut oil, wheat germ oil, peach stone oil and the liquid parts of coconut oil. Other triglyceride oils, such as the liquid fractions of beef tallow as well as synthetic triglyceride oils like coco caprylate are also suitable, however,
liquid paraffin oils, isoparaffin oils and synthetic hydrocarbons as well as di-n-alkyl ethers that include a total of 12 to 36 carbon atoms, particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert-butyl ether, diisopentyl ether, di-3-ethyldecyl ether, tert-butyl n-octyl ether, isopentyl n-octyl ether and 2-methylpentyl n-octyl ether. The compounds available as the commercial products 1,3-di-(2-ethylhexyl)cyclohexane (Cetiol® S) and di-n-octyl ether (Cetiol® OE) can be preferred.

Exemplary natural oils include amaranth seed oil, apricot stone oil, argan oil, avocado oil, babassu oil, cotton seed oil, borage seed oil, camelina oil, safflower oil, peanut oil, pomegranate stone oil, grapefruit seed oil, hemp oil, hazelnut oil, elderberry seed oil, blackcurrent seed oil, jojoba oil, cocoa butter, linseed oil, macadamia nut oil, maize seed oil, almond oil, marula oil, evening primrose oil, olive oil, palm oil, rape seed oil, rice oil, Hippophae Rhamnoides oil, sea buckthorn seed oil, sesame oil, shea butter, soya oil, sunflower oil, grape seed oil, walnut oil or wild rose oil.

Of the abovementioned ester oils, amaranth seed oil, apricot stone oil, argan oil, marula oil, macadamia oil, almond oil, in particular sweet almond oil, sesame oil, sunflower oil, olive oil, orange oil, cape chestnut oil, wheat germ oil, peach stone oil, isopropyl myristate, dicaprylyl carbonate and/or coco caprylate as well as their mixtures are particularly preferred.

These particularly preferred ester oils are used in the agents according to the invention in an amount of 0.01 to 20 wt %, preferably 0.01 to 10.0 wt %, particularly preferably 0.01 to 7.5 wt %, most highly preferably 0.1 to 5.0 wt %. Of course it is inventively possible to also use a plurality of ester oils at the same time.

Of course the hair treatment agents according to the invention also comprise, besides the active substance combination according to the invention, additional ingredients that are usual in cosmetic compositions. The choice of these ingredients is generally guided according to the intended use of the hair treatment agent. In the case of a shampoo, additional surface-active substances are comprised, for example. In the case of hair cures, additional cationic compounds and additional care products are optionally comprised. In many cases the compositions comprise at least one surface-active substance, wherein, in principle, not only anionic, but also zwitterionic, ampholytic, non-ionic and cationic surface-active substances are suitable. The choice of the surface active substances depends on the type of the agent.

Suitable anionic surfactants (Tanion) for the inventive preparations are all anionic surface-active materials that are suitable for use on the human body. Typical examples of anionic surfactants are:
linear and branched fatty acids with 8 to 30 carbon atoms (soaps),
ether carboxylic acids of the formula R—O—(CH₂—CH₂O)ₓ—CH₂—COOH, in which R is a linear alkyl group with 8 to 30 carbon atoms and x=0 or 1 to 16, and their salts,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates that include 8 to 24 carbon atoms,
linear alpha-olefin sulfonates that include 8 to 24 carbon atoms,
alpha-sulfo fatty acid methyl esters of fatty acids that include 8 to 30 carbon atoms,
alkyl sulfates and alkyl polyglycol ether sulfates of the Formula R—O(CH₂—CH₂O)ₓ—OSO₃H, in which R is preferably a linear alkyl group that includes 8 to 30 carbon atoms and x=0 or 1 to 12,
hydroxy sulfonates essentially corresponding to at least one of the two following formulas or their mixtures as well as their salts CH₃—(CH₂)_{y}—CHOH—(CH₂)ₚ—(CH—SO₃M)-(CH₂)ₙ—CH₂—O—(CₙH₂ₙO)ₓ—H, and/or CH₃—(CH₂)_{y}—(CH—SO₃M)-(CH₂)ₚ—CHOH—(CH₂)ₙ—CH₂—O—(CₙH₂ₙO)ₓ—H wherein in both Formulas y and z=0 or whole numbers from 1 to 18, p=0, 1 or 2 and the sum (y+z+p) is a number from 12 to 18, x=0 or a number from 1 to 30 and n is a whole number from 2 to 4 as well as M=H or alkali metal, in particular sodium, potassium, lithium, alkaline earth, in particular magnesium, calcium, zinc and/or an ammonium ion that can optionally be substituted, in particular mono, di, tri or tetraammonium ions with C1 to C4 alkyl, alkenyl or aryl residues,
sulfated hydroxyalkyl polyethylene glycol ethers and/or hydroxyalkylene propylene glycol ethers of the Formula R¹—(CHOSO₃M)-CHR³—(OCHR⁴—CH₂)ₙ—OR² with R¹ a linear alkyl residue with 1 to 24 carbon atoms, R² for a linear or branched, saturated alkyl residue with 1 to 24 carbon atoms, R³ for hydrogen or a linear alkyl residue with 1 to 24 carbon atoms, R⁴ for hydrogen or a methyl residue and M for hydrogen, ammonium, alkylammonium, alkanolammonium, in which the alkyl and alkanol residues each have 1 to 4 carbon atoms, or a metal atom selected from lithium, sodium, potassium, calcium or magnesium and n for a number in the range of 0 to 12 and additionally the total number of carbon atoms comprised in R¹ and R³ ranges from 2 to 44,
sulfonates of unsaturated fatty acids with 8 to 24 carbon atoms and 1 to 6 double bonds,
esters of tartaric acid and citric acid with alcohols, which represent the addition products of about 2-15 molecules of ethylene oxide and/or propylene oxide on fatty alcohols that include 8 to 22 carbon atoms,
alkyl and/or alkenyl ether phosphates of Formula (II), R¹(OCH₂CH₂)ₙ—O—(PO—OX)—OR²,
in which R¹ preferably stands for an aliphatic hydrocarbon residue with 8 to 30 carbon atoms, R² stands for hydrogen, a residue (CH₂CH₂O)ₙR² or X, n for numbers between 1 and 10 and X for hydrogen, an alkali or alkaline earth metal or NR³R⁴R⁵R⁶, with R³ to R⁶, independently of each other standing for a C₁ to C₄ hydrocarbon residue,
sulfated fatty acid alkylene glycol esters of the formula RCO(AlkO)ₙSO₃M in which RCO— stands for a linear or branched, aliphatic, saturated and/or unsaturated acyl residue with 6 to 22 carbon atoms, Alk for CH₂CH₂, CHCH₃CH₂ and/or CH₂CHCH₃, n for numbers from 0.5 to 5 and M for a metal, such as alkali metals, in particular sodium, potassium, lithium, alkaline earth metals, in particular magnesium, calcium, zinc, or ammonium ion, such as ⁺NR³R⁴R⁵R⁶ with R³ to R⁶ independently of each other standing for hydrogen or a C1 to C4 hydrocarbon residue,
monoglyceride sulfates and monoglyceride ether sulfates of the formula R⁸OC—(OCH₂CH₂)ₓ—OCH₂—[CHO(CH₂CH₂O)_{y}H]—CH₂—O—(CH₂CH₂O)_{z}—SO₃X, in which R⁸ stands for a linear or branched acyl residue that includes 6 to 22 carbon atoms, the sum of x, y and z is 0 or stands for numbers between 1 and 30, preferably 2 to 10, and X stands for an alkali metal or alkaline earth metal. In the context of the invention, typical examples of suitable monoglyceride (ether) sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride as well as their ethylene oxide adducts with sulfur trioxide or chlorosulfonic acid in the form of their sodium salts. Preferably, monoglyceride sulfates are employed, in which R⁸CO stands for a linear acyl residue that includes 8 to 18 carbon atoms,
amide ether carboxylic acids, R¹—CO—NR²—CH₂CH₂—O—(CH₂CH₂O)ₙCH₂COOM, with R¹ as a straight chain or branched alkyl or alkenyl residue with 2 to 30 carbon atoms in the chain, n stands for a whole number of 1 to 20 and R² stands for hydrogen, a methyl, ethyl, propyl, isopropyl, n-butyl, t-butyl or isobutyl residue and m stands for hydrogen or for a metal, such as alkali metals, in particular sodium, potassium, lithium, alkaline earth metals, in particular magnesium, calcium, zinc, or an ammonium ion, such as ⁺NR³R⁴R⁵R⁶, with R³ to R⁶ independently of each other standing for hydrogen or a C1 to C4 hydrocarbon residue. Such products are available for example from Chem-Y under the product name Akypo®.
acylglutamates of the Formula XOOC—CH2CH2CH(C(NH)OR)—COOX, in which RCO stands for a linear or branched acyl residue with 6 to 22 carbon atoms and 0 and/or 1, 2 or 3 double bonds and X stands for hydrogen, an alkali metal and/or alkaline earth metal, ammonium, alkylammonium, alkanolammonium or glucammonium,
condensation products of a water-soluble salt of a water-soluble albumin hydrolysate with a C8-C30 fatty acid. Such products have long been commercially available under the trade names Lamepon®, Maypon®, Gluadin®, Hostapon® KCG or Amisoft®.
carboxylates, sulfates, phosphates and/or isethionates of alkyl and/or alkenyl oligoglycosides,
acyl lactylates and
hydroxy mixed ether sulfates.

In so far as the mild anionic surfactants comprise polyglycol ether chains, the latter quite particularly preferably have a narrow homolog distribution. Furthermore, in the case of mild anionic surfactants with polyglycol ether units, the number of the glycol ether groups is preferably 1 to 20, preferably 2 to 15, particularly preferably 2 to 12. Particularly mild anionic surfactants with polyglycol ether groups without restricted homolog distribution can also be obtained for example if on the one hand the number of the polyglycol ether groups is 4 to 12 and Zn or Mg ions are selected as the counter ion. An example of this is the commercial product Texapon® ASV.

Particularly suitable zwitterionic surfactants are the so-called betaines such as the N-alkyl-N,N-dimethylammonium glycinates, for example the cocoalkyldimethylammonium glycinate, N-acylaminopropyl-N,N-dimethylammonium glycinates, for example the cocoacylaminopropyldimethylammonium glycinate, and 2-alkyl-3-carboxymethyl-3-hydroxyethyl imidazolines with 8 to 18 carbon atoms in each of the alkyl or acyl groups, as well as cocoacylaminoethylhydroxyethyl carboxymethyl glycinate. A preferred zwitterionic surfactant is the fatty acid amide derivative, known under the INCI name Cocamidopropyl Betaine.

Ampholytic surfactants (Tampho) are understood to mean those surface-active compounds that are capable of forming inner salts. Examples of suitable ampholytic surfactants are N-alkyl glycines, N-alkylamino propionic acids, N-alkylamino butyric acids, N-alkylimino dipropionic acids, N-hydroxyethyl-N-alkylamidopropylglycine, N-alkyl taurines, N-alkyl sarcosines, 2-alkylamino propionic acids and alkylamino acetic acids, each with about 8 to 24 carbon atoms in the alkyl group. Typical examples of amphoteric or zwitterionic surfactants are alkyl betaines, alkylamido betaines, amino propionates, amino glycinates, imidazolinium betaines and sulfo betaines. Particularly preferred ampholytic surfactants are N-cocoa alkylamino propionate, cocoa acylaminoethylamino propionate and C₁₂-C₁₈ acyl sarcosine.

Non-ionic surfactants (Tnio) are for example
addition products of 2 to 50 mol ethylene oxide and/or 0 to 5 mol propylene oxide to linear and branched fatty alcohols that includes 6 to 30 carbon atoms, the fatty alcohol polyglycol ethers or the fatty alcohol polypropylene glycol ethers or mixed fatty alcohol polyethers,
addition products of 2 to 50 mol ethylene oxide and/or 0 to 5 mol propylene oxide to linear and branched fatty acids that includes 6 to 30 carbon atoms, the fatty acid polyglycol ethers or the fatty acid polypropylene glycol ethers or mixed fatty acid polyethers,
addition products of 2 to 50 mol ethylene oxide and/or 0 to 5 mol propylene oxide to linear and branched alkylphenols that include 8 to 15 carbon atoms in the alkyl group, the alkylphenol polyglycol ethers or the alkylphenol polypropylene glycol ethers or mixed alkylphenol polyethers,
methyl or C₂-C₆ alkyl residue end blocked addition products of 2 to 50 mol ethylene oxide and/or 0 to 5 mol propylene oxide to linear and branched fatty alcohols with 8 to 30 carbon atoms, to fatty acids with 8 to 30 carbon atoms and to alkylphenols with 8 to 15 carbon atoms in the alkyl group, such as, for example, the available types under the sales names Dehydrol® LS, Dehydrol® LT (Cognis),
C₁₂-C₃₀ fatty acid mono and diesters of addition products of 1 to 30 mol ethylene oxide to glycerin,
addition products of 5 to 60 mol ethylene oxide to castor oil and hydrogenated castor oil, polyol esters of fatty acids, such as, for example, the commercial product Hydagen® HSP (Cognis) or Sovermol types (Cognis),
alkoxylated triglycerides,
alkoxylated fatty acid alkyl esters of the formula (Tnio-1) R¹CO—(OCH₂CHR²)_{w}OR³ (Tnio-1) in which R¹CO stands for a linear or branched, saturated and/or unsaturated acyl residue that includes 6 to 22 carbon atoms, R² for hydrogen or methyl, R³ for linear or branched alkyl residues that include 1 to 4 carbon atoms and w for numbers from 1 to 20,
amine oxides,
hydroxy mixed ethers,
R¹O[CH₂CH(CH₃)O]ₓ(CH₂CHR²O)_{y}[CH₂CH(OH)R³]_{z} with R¹ standing for a linear or branched, saturated or unsaturated alkyl and/or alkenyl residue that includes 2 to 30 carbon atoms, R² stands for hydrogen, a methyl, ethyl, propyl or isopropyl residue, R³ stands for a linear or branched alkyl residue that includes 2 to 30 carbon atoms, x stands for 0 or a number from 1 to 20, y for a number from 1 to 30 and z stands for the number 1, 2, 3, 4 or 5.
sorbitol esters of fatty acids and addition products of ethylene oxide to sorbitol esters of fatty acids such as e.g. the polysorbates,
sugar esters of fatty acids and addition products of ethylene oxide to sugar esters of fatty acids,
addition products of ethylene oxide to fatty acid alkanolamides and fatty amines,
sugar surfactants of the alkyl and alkenyl oligoglycoside type,
sugar surfactants of the fatty acid N-alkylpolyhydroxyalkylamide type,
fatty acid amide polyglycol ethers, fatty amine polyglycol ethers,
mixed ethers or mixed formals and polysorbates.

Cationic surfactants of the Formula (Tkat1-1) can be additionally used.

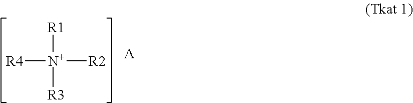

In the Formula (Tkat1), R1, R2, R3 and R4 each stand independently of each other for hydrogen, a methyl group, a phenyl group, a benzyl group, for a saturated, branched or unbranched alkyl residue with a chain length of 8 to 30 carbon atoms which can be substituted with one or more hydroxy groups. A stands for a physiologically acceptable anion, for example halides such as chloride or bromide as well as methosulfates.

Exemplary compounds of the Formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate.

The surfactants (T) are employed in quantities of 0.05 to 45 wt %, preferably 0.1 to 30 wt % and quite particularly preferably from 0.5-25 wt %, based on the total inventively used agent.

Exemplary inventively usable emulsifiers are
addition products of 4 to 30 mol ethylene oxide and/or 0 to 5 mol propylene oxide to linear fatty alcohols that include 8 to 22 carbon atoms, to fatty acids that include 12 to 22 carbon atoms and to alkyl phenols that include 8 to 15 carbon atoms in the alkyl group,
C₁₂-C₂₂ fatty acid mono- and diesters of addition products of 1 to 30 mol ethylene oxide on polyols that include 3 to 6 carbon atoms, especially on glycerin,
ethylene oxide- and polyglycerin-addition products on methyl glucoside fatty acid esters, fatty acid alkanolamides and fatty acid glucamides,
C₈-C₂₂ alkyl monoglycosides and -oligoglycosides and their ethoxylated analogs, wherein the degrees of oligomerization are 1.1 to 5, particularly 1.2 to 2.0, and glucose as the sugar component are preferred,
mixtures of alkyl(oligo)glucosides and fatty alcohols, for example the commercial product Montanov® 68,
addition products of 5 to 60 mol ethylene oxide to castor oil and hydrogenated castor oil,
partial esters of polyols that include 3-6 carbon atoms with saturated fatty acids that include 8 to 22 carbon atoms,
sterols, both from animal tissue (zoosterols, cholesterols, lanosterols) as well as from vegetal fats (phytosterols, ergosterol, stigmasterol, sitosterol) or from fungi and yeasts (mycosterols),
phospholipids (lecithines, phosphatidyl cholines),
fatty acid esters of sugars and sugar alcohols, such as sorbitol,
polyglycerins and polyglycerin derivatives such as for example polyglycerin poly-12-hydroxystearate (commercial product Dehymuls® PGPH).

The inventive agents preferably comprise the emulsifiers in quantities of 0.1 to 25 wt %, particularly 0.5-15 wt %, based on the total agent.

The compositions according to the invention particularly preferably comprise fats as the additional active principle. Fats are understood to mean fatty acids, fatty alcohols, natural and synthetic waxes that can exist both in solid form as well as liquid in aqueous dispersion, and natural and synthetic cosmetic oil components.

Linear and/or branched, saturated and/or unsaturated fatty acids that include 6-30 carbon atoms can be employed as the fatty acids (Fatac). Fatty acids having 10-22 carbon atoms are preferred. Among these may be cited the isostearic acids, such as the commercial products Emersol® 871 and Emersol® 875, and isopalmitic acids such as the commercial product Edenor® IP 95, as well as all other fatty acids commercialized under the trade names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Usually, the fatty acid fractions obtainable from coconut oil and palm oil are particularly preferred; in general, the addition of stearic acid is particularly preferred.

The addition quantity ranges from 0.1-15 wt %, based on the total agent. The quantity preferably ranges from 0.5-10 wt %, wherein quantities of 1-5 wt % can be quite particularly advantageous.

As fatty alcohols Fatal), saturated, mono or polyunsaturated, branched or linear fatty alcohols that include C₆ to C₃₀, preferably C₁₀ to C₂₂ and quite particularly preferably C₁₂ to C₂₂ carbon atoms can be added. In the scope of the invention, decanol, octanol, octenol, dodecenol, decenol, octadienol, dodecadienol, decadienol, oleyl alcohol, eruca alcohol, ricinyl alcohol, stearyl alcohol, isostearyl alcohol, cetyl alcohol, lauryl alcohol, myristyl alcohol, arachidyl alcohol, capryl alcohol, caprinic alcohol, linalyl alcohol, linolenyl alcohol and behenyl alcohol, as well as the Guerbet alcohols can be added, this listing being intended as exemplary and not limiting in character. However, the fatty alcohols are preferably derived from naturally occurring fatty acids, usually obtained by reducing the fatty acid esters. Those fatty alcohol fractions that represent a mixture of different fatty alcohols are likewise inventively employable. Such substances can be bought, for example, under the trade names Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, wool wax alcohols such as those that are commercially available for example, under the trade names Corona®, White Swan®, Coronet® or Fluilan® can also be inventively added. The fatty alcohols are added in quantities of 0.1-30 wt %, based on the total preparation, preferably in quantities of 0.1-20 wt %.

According to the invention, solid paraffins or isoparaffins, carnuba wax, bean wax, candelilla wax, ozocerite, ceresine, sperm wax, sunflower wax, fruit waxes such as for example apple wax or citrus wax, microwaxes of PE or PP can be added as the natural or synthetic waxes. These types of waxes are available, for example, from Kahl & Co., Trittau.

The added quantities are 0.1 to 50% by weight, based on the total agent, preferably 0.1 to 20% by weight and particularly preferably 0.1 to 15% by weight, based on the total agent.

The total amount of oil and fat components in the inventive agents is normally 0.5-75 wt %, based on the total agent. Quantities of 0.5 to 35 wt % are inventively preferred.

Vitamins, provitamins or vitamin precursors are a further preferred group of ingredients of the inventive compositions with the inventive active principle complex. Here, vitamins, provitamins and vitamin precursors which are assigned to the groups A, B, C, E, F and H are particularly preferred.

The group of substances designated as vitamin A includes retinol (vitamin A_{I}) as well as 3,4-didehydroretinol (vitamin A₂). β-Carotene is the provitamin of retinol. Examples of suitable vitamin A components according to the invention are vitamin A acid and its esters, vitamin A aldehyde and vitamin A alcohol as well as its esters such as the palmitate and acetate. The agents according to the invention preferably comprise the vitamin A components in amounts of 0.05 to 1 wt %, based on the total preparation.

The vitamin B group or the vitamin B complex include inter alia:

Vitamin B₁ (Thiamine)

Vitamin B₂ (Riboflavin)

Vitamin B₃. The compounds nicotinic acid and nicotinamide (niacinamide) are often included under this designation. According to the invention, nicotinamide is preferred and is comprised in the agents used according to the invention in amounts of 0.05 to 1 wt %, based on the total agent.

Vitamin B₅ (pantothenic acid, panthenol and pantolactone). In the context of this group, panthenol and/or pantolactone is preferably used. Useable derivatives of panthenol according to the invention are especially the esters and ethers of panthenol as well as cationically derivatized panthenols. Specific representatives are for example, panthenol triacetate, panthenol monoethyl ether and its monoacetate as well as cationic panthenol derivatives. In the present invention, pantothenic acid is preferably employed as a derivative in the form of the stabilized calcium salt and sodium salt (Ca pantothenate, Na pantothenate).

Vitamin B₆ (pyridoxine as well as pyridoxamine and pyridoxal).

The cited compounds of the vitamin B type, in particular vitamin B₃, B₅ and B₆, are preferably comprised in the agents according to the invention in amounts of 0.05 to 10 wt %, based on the total agent. Quantities of 0.1 to 5 wt % are particularly preferred.

Vitamin C (ascorbic acid). Vitamin C is preferably added to the agents according to the invention in amounts of 0.1 to 3 wt %, based on the total agent. Its use in the form of the palmitate ester, the glucosides or phosphates can be preferred. The use in combination with tocopherols can also be preferred.

Vitamin E (Tocopherols, especially α-tocopherol). Tocopherol and its derivatives, among which are particularly included the esters such as the acetate, the nicotinate, the phosphate and the succinate, are used in the agents according to the invention preferably comprised in amounts of 0.05-1 wt %, based on the total agent.

Vitamin F. The term “vitamin F” is usually taken to mean essential fatty acids, particularly linoleic acid, linolenic acid and arachidonic acid.

Vitamin H. The compound (3aS,4S,6aR)-2-oxohexahydrothienol[3,4-d]imidazole-4-valeric acid denotes Vitamin H, for which the trivial name biotin has become accepted. The agents according to the invention preferably comprise biotin in amounts of 0.0001 to 1.0 wt %, particularly in amounts of 0.001 to 0.01 wt %.

The compositions according to the invention preferably comprise vitamins, provitamins and vitamin precursors from the groups A, B, E and H. Panthenol, pantolactone, pyridoxine and its derivatives as well as nicotinamide and biotin are especially preferred.

A particularly preferred group of ingredients in the cosmetic compositions according to the invention are the betaines cited below: carnitine, carnitine tartrate, carnitine magnesium citrate, acetylcarnitine, betalaine, 1,1-dimethylproline, choline, choline chloride, choline bitartrate, choline dihydrogen citrate and the compound N,N,N-trimethylglycine cited in the literature as a betaine.

Carnitine, histidine, choline as well as betaine are preferably used. In a particularly preferred embodiment of the invention, L-carnitine tartrate is employed as the active principle.

In another inventively preferred embodiment, the inventive compositions comprise bioquinones. Suitable bioquinones in the inventive agents are understood to include one or more ubiquinones and/or plastoquinones. The preferred ubiquinones according to the invention have the following formula:

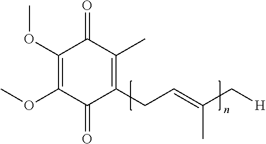

with n=6, 7, 8, 9 or 10.

The coenzyme Q-10 is most preferred here.

Preferred compositions according to the invention comprise purine and/or purine derivatives in narrow quantitative ranges. Inventively preferred cosmetic agents are characterized in that they comprise, based on their weight, 0.001 to 2.5 wt %, preferably 0.0025 to 1 wt %, particularly preferably 0.005 to 0.5 wt % and particularly 0.01 to 0.1 wt % purine and/or purine derivative(s). Inventively preferred cosmetic agents are characterized in that they comprise purine, adenine, guanine, uric acid, hypoxanthine, 6-purinethiol, 6-thioguanine, xanthin, caffein, theobromine or theophylline. Caffein is most preferred in hair cosmetic preparations.

In another preferred embodiment of the present invention, the cosmetic agent comprises ectoine ((S)-2-methyl-1,4,5,6-tetrahydro-4-pyrimidine carboxylic acid).

Agents that comprise, based on their weight, 0.00001 to 10.0 wt %, preferably 0.0001 to 5.0 wt % and particularly 0.001 to 3 wt % of the active principles from the group made up of carnitine, coenzyme Q-10, ectoine, a vitamin of the B series, a purine and their derivatives or physiologically representative salts are inventively particularly preferred.

A quite particularly preferred care additive in the hair treatment agents according to the invention is taurine. Taurine is understood to mean exclusively 2-aminoethane sulfonic acid and by a derivative the explicitly cited derivatives of taurine. The derivates of taurine are understood to mean N-monomethyltaurine, N,N-dimethyltaurine, taurine lysylate, taurine tartrate, taurine ornithate, lysyltaurine and ornithyltaurine.

Inventively particularly preferred agents according to the invention comprise, based on their weight, 0.0001 to 10.0 wt %, preferably 0.0005 to 5.0 wt %, particularly preferably 0.001 to 2.0 wt % and particularly 0.001 to 1.0 wt % of taurine and/or a derivative of taurine. Moreover, the action of the compositions according to the invention can be further augmented by a 2-pyrrolidone-5-carboxylic acid and its derivatives (J). The sodium, potassium, calcium, magnesium or ammonium salts are preferred, in which the ammonium ion carries one to three C₁ to C₄ alkyl groups besides hydrogen. The sodium salt is quite particularly preferred. The quantities employed in the inventive agents preferably range from 0.05 to 10 wt %, based on the total composition, particularly preferably 0.1 to 5, and particularly 0.1 to 3 wt %.

The use of plant extracts as the care substances allows the hair treatment agents according to the invention to be formulated particularly in harmony with nature and nevertheless very effectively in regard to their care performance. The otherwise usual preservatives can even be optionally obviated. Above all the inventively preferred extracts are from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow, meristem, ginseng, coffee, cocoa, moringa, ginger and Ayuvedic plant extracts such as for example Aegle Marmelos (Bilwa), Cyperus Rotundus (Nagar Motha), Emblica Officinalis (Amalki), Morida Citrifolia (Ashyuka), Tinospora Cordifolia (Guduchi), Santalum album, (Chandana), Crocus Sativus (Kumkuma), Cinnamonum Zeylanicum and Nelumbo Nucifera (Kamala), sweet grasses like wheat, barley, rye, oats, spelt, maize, the various varieties of millet (sorghum, crabgrass, foxtail millet as examples), sugar cane, meadow fescue, meadow foxtail, oat grass, bent grass, tall grass, moor grass, bamboo, cotton grass, fountain grasses, Andropogonodeae (Imperata Cylindrica also called cogon grass), buffalo grass, cord grass, Bermuda grasses, love grass, Cymbopogon (lemon grass), Oryzeae (rice), Zizania (wild rice), marram grass, blue oat, creeping soft grass, quaking grasses, meadow grasses, wheatgrasses and Echinacea purpurea (L.) Moench, all types of wine as well as pericarp of Litchie chinensis.

According to the invention, the plant extracts can be used in pure as well as in diluted form. When they are used in diluted form, they normally comprise ca. 2-80 wt % active substance and the solvent is the extraction agent or mixture of extraction agents used for their extraction.

Occasionally, it may be required to use anionic polymers. Exemplary anionic monomers, from which such polymers can be made, are acrylic acid, methacrylic acid, crotonic acid, maleic anhydride and 2-acrylamido-2-methylpropane sulfonic acid. Here, the acidic groups may be wholly or partially present as sodium, potassium, ammonium, mono- or triethanolammonium salts. Preferred monomers are 2-acrylamido-2-methylpropane sulfonic acid and acrylic acid.

Anionic polymers that comprise 2-acrylamido-2-methylpropane sulfonic acid alone or as the comonomer, have proven to be quite particularly effective; the sulfonic acid group may be fully or partially present as the sodium, potassium, ammonium, mono- or triethanolammonium salt.

The homopolymer of 2-acrylamido-2-methylpropane sulfonic acid, which is commercially available, for example under the trade name Rheothik® 11-80, is particularly preferred.

Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers and particularly polyacrylamide copolymers with monomers that include sulfonic acid groups. Such a polymer is comprised in the commercial product Sepigel® 305 from the SEPPIC company.

Likewise preferred anionic homopolymers are uncrosslinked and crosslinked polyacrylic acids. Preferred crosslinking agents can be allyl ethers of pentaerythritol, of sucrose and of propylene. Such compounds are commercially available under the trade name Carbopol®, for example.

Copolymers of maleic anhydride and methyl vinyl ether, especially those with crosslinks are also color-conserving polymers. A maleic acid-methyl vinyl ether copolymer crosslinked with 1,9-decadiene is commercially available under the trade name Stabileze® QM.

The compositions according to the invention preferably comprise the anionic polymers in quantities of 0.05 to 10 wt %, based on the total composition. Quantities of 0.1 to 5 wt % are particularly preferred.

In a further embodiment, the inventive agents can comprise non-ionic polymers.

Suitable non-ionic polymers are, for example:
vinyl pyrrolidone-vinyl ester copolymers, such as, for example, those marketed by BASF under the trade name Luviskol®, Luviskol® VA 64 and Luviskol® VA 73, each vinyl pyrrolidone-vinyl acetate copolymers, are likewise preferred non-ionic polymers.
cellulose ethers, such as hydroxypropyl cellulose, hydroxyethyl cellulose, and methyl hydroxypropyl cellulose, as marketed for example under the trademarks Culminal® and Benecel® (AQUALON) and Natrosol® types (Hercules).
starch and its derivatives, especially starch ethers, for example Structure® XL (National Starch), a multifunctional, salt tolerant starch;
shellac
polyvinyl pyrrolidones, as are marketed, for example, under the name Luviskol® (BASF).

The compositions according to the invention preferably comprise the non-ionic polymers in quantities of 0.05 to 10 wt %, based on the total composition. Quantities of 0.1 to 5 wt % are particularly preferred.

In another embodiment, the compositions according to the invention additionally comprise at least one UV light filter. UVB filters can be oil-soluble or water-soluble.

As oil-soluble substances, the following may be cited, for example:
3-benzylidene camphor, e.g. 3-(4-methylbenzylidene) camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (Octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as e.g. 2,4,6-trianilino-(p-carbo-2′-ethyl-1′-hexyloxy)-1,3,5-triazine and octyl triazone.
propane-1,3-diones, such as e.g. 1-(4-tert-butylphenyl)-3-(4′-methoxyphenyl)propane-1,3-dione;

Water-soluble substances include:
2-phenylbenzimidazole-5-sulfonic acid and its alkali metal, alkaline earth metal, ammonium, alkylammonium, alkanolammonium and glucammonium salts;
sulfonic acid derivatives of benzophenones, preferably 2-hydroxy-4-methoxybenzophenone-5-sulfonic acid and its salts;
sulfonic acid derivatives of 3-benzyldenecamphor, such as e.g. 4-(2-oxo-3-bornylidenemethyl)benzene sulfonic acid and 2-methyl-5-(2-oxo-3-bornylidene)sulfonic acid and their salts.

Typical UV-A filters particularly concern derivatives of benzoylmethane, such as, for example 1-(4′-tert-butylphenyl)-3-(4′-methoxyphenyl)propane-1,3-dione or 1-phenyl-3-(4′-isopropylphenyl)propane-1,3-dione. Naturally, the UV-A and UV-B filters can also be added as mixtures. Beside the cited soluble materials, insoluble pigments, namely finely dispersed metal oxides or salts can also be considered for this purpose, such as for example titanium dioxide, zinc oxide, iron oxide, aluminum oxide, cerium oxide, zirconium oxide, silicates (talc), barium sulfate and zinc stearate. Here, the particles should have a mean diameter of less than 100 nm, preferably between 5 and 50 nm and especially between 15 and 30 nm. They can be spherical, however elliptical or other non-spherical shaped particles can also be used.

Exemplary additional active substances, adjuvants and additives, which can be comprised in the cosmetic agents, are:
structurants such as maleic acid and lactic acid,
swelling agents such as urea, allantoin, carbonates or hydantoin,
dimethyl isosorbitol and cyclodextrins,
dyestuffs to color the agent,
anti-dandruff active materials such as Piroctone Olamine, zinc Omadine and Climbazole,
chelating agents such as EDTA, NTA, β-alanine diacetic acid and phosphonic acids,
opacifiers such as latex, styrene/PVP copolymers and styrene/acrylamide copolymers
pearlizing compositions such as ethylene glycol mono- and distearate as well as PEG-3 distearate,
pigments,
stabilizers for hydrogen peroxide and other oxidizing agents,
propellants such as propane-butane mixtures, N₂O, dimethyl ether, CO₂ and air,
antioxidants,
perfume oils, scents and fragrances.

With regard to further optional components and their amounts used of these components, reference is expressly made to the relevant handbooks known to the person skilled in the art.

Consequently, another subject matter of the invention is a method for treating hair, in which a hair treatment agent according to claim 1 is applied onto the hair and rinsed out of the hair after a contact time.

The contact time is preferably a few seconds to 100 minutes, particularly preferably 1 to 50 minutes and quite particularly preferably 1 to 30 minutes.

A method, in which a cosmetic agent according to claim 1 is applied onto the hair and remains there, is also inventive. “To remain on the hair” is inventively understood to mean that the agent, after its application, is not immediately rinsed out of the hair again. In fact in this case the agent remains for more than 100 minutes on the hair until the next hair wash.

The following examples are intended to illustrate the subject matter of the invention in more detail, without limiting it in any way.

### EXAMPLES

All indications of quantities are parts by weight unless otherwise stated. The following formulations were prepared using known production methods

| Inventive shampoo | S1 |
|---|---|
| Sodium Laureth-2 sulfate | 10.5 |
| Laureth-2 | 0.15 |
| PEG-55 propylene glycol oleate | 0.15 |
| Disodium Cocoamphoacetate | 6.0 |
| Polyquaternium-10 | 0.5 |
| PEG-7 Glyceryl Cocoate | 3.0 |
| Panthenol | 0.2 |
| Niacinamide | 0.1 |
| PEG-40 Hydrogenated Castor Oil | 0.5 |
| Laurdimonium Hydroxypropyl Hydrolyzed Wheat Protein | 0.5 |
| Polymer acc. b) claim 1, e.g. Crodasone ® W, | 0.65 |
| or Keravis ® PE | |
| Propylene Glycole Dibenzoate | 1.0 |
| Protein hydrolysate (Nutrilan I, or Gluadin W40, | 0.5 |
| or Nutrilan Keratin, pure or in mixtures) | |
| pH adjustment with citric acid, tartaric acid | Up to pH |
| and their salts and opt. NaOH or KOH) | 4.5 to 5.8 |
| Preservative (phenoxyethanol, parabene, | 0.4 |
| sorbate and their mixtures) | |
| Perfume | 0.5 |
| Water | ad 100 |

| Inventive conditioner | C1 |
|---|---|
| Isopropyl myristate | 1.0 |
| Glycerin monostearate | 0.8 |
| Quaternium-87 | 2.5 |
| Dipalmitoyl Hydroxyethylmonium Methosulfate | 1.0 |
| Cetearyl Alcohol | 5.0 |
| Ceteareth-20 | 0.6 |
| Stearamidopropyldimethylamine | 0.8 |
| Polyquaternium-37 | 0.6 |
| Dimethicone (and) Dimethiconol | 2.0 |
| Panthenol | 0.2 |
| Niacinamide | 0.1 |
| Laurdimonium Hydroxypropyl Hydrolyzed Wheat Protein | 0.5 |
| Polymer acc. b) claim 1, e.g. Crodasone ® W, | 0.65 |
| or Keravis ® PE | |
| Propylene Glycol Dibenzoates | 1.0 |
| Protein hydrolysate (Nutrilan I, or Gluadin W40, | 0.5 |
| or Nutrilan Keratin, pure or in mixtures) | |
| pH adjustment with citric acid, tartaric acid | Up to pH |
| and their salts and opt. NaOH or KOH) | 2.0 to 4.8 |
| Preservative (phenoxyethanol, parabene, | 0.4 |
| sorbate and their mixtures) | |
| Perfume | 0.5 |
| Water | ad 100 |

The viscosities of both of the above compositions are between 600 and 40 000 mPas measured according to Brookfield at 25° C. All compositions can be filled into a usual shaking flask or into pots. However, they can also packaged in an appropriate pump package equipped with a usual dosing pump. In this regard, the dosing pump can of course comprise a sieve, such that as air is mixed with the composition in the pump head, foam is discharged.

Finally, the aerosol cans can be filled together with usual propellants such as isopropane, propane, isobutane, butane, tert-butane, pentane, isopentane, tent-pentane, neopentane or dimethyl ether as well as any mixtures thereof in the ratio 90:10, preferably 92:8 (propellant). In this case as well, a particularly creamy and stable foam is obtained, which can be very well dispersed in the hair.

The technical application assessment was carried out on each of 20 heads by 5 hairdressers in the so-called half-side test. The formulations S1 and C1 were tested against the formulations VS1 to VC1. The formulations VS1 to VC1 are the formulations S1 and C1 but without polymer b) according to claim 1 and propylene glycol dibenzoates, the active substance combination according to the invention.

As usual the hair was washed with a shampoo and dried with a hand towel. The half heads were parted in the middle with a comb and 5 g of each test composition were sprayed on, i.e. S1 against VS1 etc. After a contact time of 5 minutes at about 25° C. the compositions were rinsed out again with running water at about 32° C. for 3 minutes. The comparative assessment was then carried out by the 5 hairdressers.

The inventive compositions S1 and C1 each proved to be better by at least 0.67 points for the parameters: treating the wet hair with the hairdryer, combability, feel of the wet and dry hair, sentiment of the wet and dry tips of the hair, body of the dry hair, as well as gloss of the dry hair.

In addition, the long-term effect of the obtained hairstyle was appraised. For this, the models were assessed again 24 h after the application. All inventive compositions proved to be at least 1 point better in each parameter.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for the care of keratinic fibers comprising—each relative to the total composition—
a) at least one diester of the propylene glycol according to the Formula I 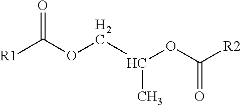
in which R1 and R2 each independently of one another stand for a residue selected from at least one linear, saturated or unsaturated, branched, saturated or unsaturated as well as cyclic saturated or unsaturated C6 to C30 hydrocarbon residue and/or a phenyl or benzyl residue that is optionally substituted with C1 to C4 alkyl groups and/or hydroxy groups as well as their mixtures in a total amount of 0.01 to 5.0 wt % and
b) at least one polymer in a total amount of 0.001 to 5.0 wt %, wherein said polymer is composed of at least one protein hydrolysate as well as at least one silane.

2. The cosmetic composition according to claim 1, further comprising at least one quaternary ammonium compound in a total amount of 0.1 to 10 wt % relative to the weight of the total composition, selected from one of the group consisting of
i) Esterquats,
ii) quaternary imidazolines of the Formula (Tkat2), 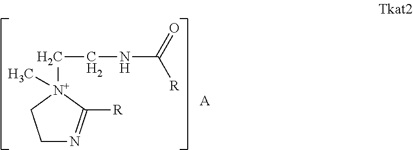
in which the R groups independently of each other each stand for a saturated or unsaturated, linear or branched hydrocarbon residue with a chain length of 8 to 30 carbon atoms and A stands for a physiologically acceptable anion,
iii) amines and/or cationized amines,
iv) poly(methacryloyloxyethyltrimethylammonium compounds),
v) quaternized cellulose derivatives,
vi) cationic alkyl polyglycosides,
vii) cationized honey,
viii) cationic guar derivatives,
ix) Chitosan,
x) polymeric dimethyldiallylammonium salts and their copolymers with esters and amides of acrylic acid and methacrylic acid,
xi) copolymers of vinyl pyrrolidone with quaternized derivatives of dialkylaminoalkyl acrylate and dialkylaminoalkyl methacrylate,
xii) vinyl pyrrolidone-vinyl imidazolium methochloride copolymers,
xiii) quaternized polyvinyl alcohol,
xiv) Polyquaternium-74,
xv) cationic alkyl oligoglucosides,
xvi) Polyquaternium-71, and
mixtures thereof.

3. The cosmetic composition according to claim 1, wherein the R1 and R2 groups in the Formula I of the diester of the propylene glycol are the same.

4. The cosmetic composition according to claim 1, wherein the R1 and R2 residues are the same and are selected from phenyl or benzyl.

5. The cosmetic composition according to claim 1, further comprising ester oils in a total amount of 0.01 to 20 wt % selected from the group consisting of amaranth seed oil, apricot kernel oil, argan oil, marula oil, macadamia oil, (sweet) almond oil, sesame oil, sunflower oil, olive oil, orange oil, cape chestnut oil, wheat germ oil, peach kernel oil, isopropyl myristate, dicapryl carbonate, coco caprylate, and mixtures thereof.

6. The cosmetic composition according to claim 1, wherein the polymer is one or more polymers selected from the group consisting of Hydrolyzed Vegetable Protein PG-Propyl Silanetriol, Hydrolyzed Wheat Protein PG-Propyl Silanetriol, and Cystine Bis-PG-Propyl Silanetriol.

7. The cosmetic composition according to claim 1, wherein the composition has a viscosity of 600 to 40 000 mPas, measured according to Brookfield at a temperature of 25° C.

8. A method for the treatment of keratinic fibers, comprising:
applying a cosmetic composition according to claim 1 onto the keratinic fibers, and rinsing out the cosmetic composition after a contact time of a few seconds up to 45 minutes.
